(19) United States
(12) Patent Application Publication
(10) Pub. No.: US 2005/0132508 A1
(43) Pub. Date: Jun. 23, 2005

(54) Dry cleaning process

(75) Inventor: Pieter Everhardus Overdevest, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 11/012,008

(22) Filed: Dec. 14, 2004

(30) Foreign Application Priority Data
Dec. 19, 2003 (EP) 03079125.5

(51) Int. Cl. A61K 7/13

(52) U.S. Cl. 8/412

(57) ABSTRACT

The invention provides an in-home dry cleaning process carried out in a dry-cleaning machine containing a non-flammable, non-chlorine containing organic dry cleaning solvent, and comprising the steps of (i) mixing the solvent with a disposable treatment composition to form a dry cleaning composition and subsequently (ii) (ii) contacting a laundry article with the dry cleaning composition. The disposable treatment composition contains a surfactant and/or cleaning agent and water in an amount which is sufficient to give both good cleaning performance and effective fabric care.

### FIELD OF THE INVENTION

The present invention relates to a dry cleaning process wherein a disposable treatment composition is used. The dry cleaning process is suitable for treating laundry articles, whereby the treatment includes the removal of undesired soil or odour. In particular, the dry cleaning process is suitable for in-home use.

### BACKGROUND OF THE INVENTION

In general, laundry articles can be cleaned using water as the primary medium with surfactants and other cleaning agents. However, some laundry articles can not be safely cleaned with water. For these a dry cleaning process may be used wherein an organic dry cleaning solvent is the primary medium. Applications involving one or more stages of immersion, rinsing and/or drying are known. Solvents can be used at ambient temperature or at elevated temperatures up to the boiling point of the solvent.

But dry cleaning is only available in specialised outlets and usually consumers have to bring and pick up their clothes which is not convenient. Some proposals have been made to towards an in-home dry cleaning process i.e., a dry cleaning process for relatively small wash loads suitable for use in domestic environments.

However, the use of an organic dry cleaning solvent in domestic environments does require a more stringent approach regarding safety and ease of use. Domestic environments are usually well adapted for aqueous washing. Water is available from a tap and can be discharged to the sewer after washing. Evidently, this will be more complicated with dry cleaning solvents. Fresh solvent needs to be supplied to replenish lost solvent. The supply of fresh solvent will probably be carried out via cylinders—which need to be purchased separately from time to time. The dry cleaning machine will probably have to be designed as a closed system. This is to retain substantially all of the solvent to minimise losses into the environment. In addition, the whole process of adding fresh solvent and collecting used solvent will also have to meet such stringent requirements. This complicates the logistics and management of an in-home dry cleaning process. On the other hand, to be carried out by the consumer, an in-home dry cleaning process should be as user friendly as conventional water based washing. The necessary steps must be simple and the process should have minimal maintenance requirements. Therefore, there is a need for a user friendly, low cost in-home dry cleaning process which still delivers effective cleaning and garment care.

### DEFINITION OF THE INVENTION

According to the invention an in-home dry cleaning process is provided said process being carried out in a dry-cleaning machine containing a non-flammable, non-chlorine containing organic dry cleaning solvent, and comprising the steps of
(i) mixing said solvent with a disposable treatment composition to form a dry cleaning composition; and subsequently
(ii) contacting a laundry article with the dry cleaning composition,
wherein said disposable treatment composition contains
(a) an effective amount of at least one surfactant and/or cleaning agent;
(b) an amount of water (expressed as ml water/kg laundry) of less than 100, preferably less than 50, more preferably less than 3, and more than 0.01, preferably more than 0.05, more preferably more than 0.1, wherein said amount of water is sufficient to give both good cleaning performance and effective fabric care.

### DETAILED DESCRIPTION OF THE INVENTION

The present invention provides a user friendly in-home dry cleaning process. It obviates or at least minimises the need for complicated and expensive in-home dry cleaning machines with water metering devices and/or water management systems while still providing effective cleaning and garment care.

As mentioned above, the process of the invention is carried out in a dry cleaning machine containing a dry cleaning solvent. This dry cleaning solvent is generally regenerated after every laundry treatment cycle in which said solvent is used. An additive—which is called disposable treatment composition in connection with the present invention—is usually added before each laundry treatment cycle for improving cleaning. In order to obtain the best possible laundry fabric care performance no water should be added. However, addition of some water is necessary to achieve the desired cleaning performance. The inventors have now established the optimum amount of water that needs to be added as part as the additive before each laundry treatment cycle, such that both good fabric care and favourable cleaning performance are obtained.

Definitions

The term “dry cleaning process” used herein is intended to mean any process wherein laundry articles are contacted with a dry cleaning composition within a closable vessel. It is to be understood that this is also meant to encompass other treatments such as but not limited to softening and refreshing. However, as used herein this term does not include any process comprising cycles wherein the laundry articles are also immersed and rinsed in an aqueous cleaning composition comprising more than 80 wt. % water because this would usually damage garments that can only be dry cleaned.

The term “dry cleaning composition” as used herein is intended to mean the composition used in the dry cleaning process including the dry cleaning solvent, any surfactant, cleaning agents but excluding the laundry articles that are to be cleaned.

The term “organic dry cleaning solvent” as used herein is intended to mean any non-aqueous solvent that preferably has a liquid phase at 20° C. and standard pressure. The term organic has its usual meaning, i.e., a compound with at least one carbon hydrogen bond.

The term “disposable treatment composition” as used herein is intended to describe a composition comprising at least one surfactant and/or cleaning agent useful for treating laundry articles.

When referring to the “weight of the cloth”, it is intended to mean the weight of the cloth of the laundry article after the cloth has been equilibrated at 20° C., a relative humidity of 55% and standard pressure.

The term “laundry article” as used herein is typically a garment but may include any textile article. Textile articles include—but are not limited to—those made from natural fibres such as cotton, wool, linen, hemp, silk and man made fibres such as nylon, viscose, acetate, polyester, polyamide, polypropylene elastomer, natural or synthetic leather, natural or synthetic fur and mixtures thereof.

The term “liquid to cloth ratio” (w/w) (LCR) as used herein is intended to mean the ratio of the weight of the total amount of dry cleaning composition to the weight of the cloth as defined above.

The term “immerse” as used herein is intended to mean that the laundry article is contacted with a cleaning effective amount of dry cleaning composition in a cycle of the dry cleaning process to wet the laundry article which is usually a LCR of greater than 0.5 or more preferably a LCR as given below.

The term “cleaning effective amount” as defined herein is intended to mean an amount effective to obtain the desired cleaning.

The term “in-home” as defined herein is intended to mean that the LCR of the dry cleaning cycle is at most 20. Although, the in-home dry cleaning is especially suitable for domestic homes, in some cases these small appliances may also be used in hotels, airports on a non-industrial scale.

The water content refers to water purposefully added to the laundry articles, for example as part of the dry cleaning composition as such or a pre-treatment composition, including hydrated water as part of ingredients making up these compositions. It is not intended to include the moisture of the untreated wash load e.g., a wet towel.

Dry Cleaning Process

The dry cleaning process may comprise different cleaning and rinsing cycles in any order depending on the desired outcome. The number and length of cycles may vary depending on the desired result. For the purpose of the present invention a rinse cycle is defined as a cycle wherein the laundry articles are agitated in dry cleaning solvent only. When surfactant and/or cleaning agent is present, the cycle is described as a cleaning cycle whereby cleaning is understood to encompass conditioning.

A cycle wherein surfactant and/or cleaning agent is used will normally comprise of different steps such as, mixing a disposable treatment composition with a dry cleaning solvent to form a dry cleaning composition, contacting a laundry article with said composition, agitating the laundry article in said composition, removing said composition from the laundry article. The removal may be carried out by any means known in the art such draining, spinning or when appropriate evaporating the composition, or any combination thereof.

Generally, articles such as clothing are cleaned by contacting a cleaning effective amount of a dry cleaning composition with the articles for an effective period of time to clean the articles or otherwise remove stains. Each cycle may preferably last from at least 0.1 min, or preferably at least 0.5 min or more preferably at least 1 min or even 5 min, and at most 2 hrs, preferably at most 30 min, even more preferably at most 20 min and in some instances at most 5 min. In some cases longer times may be desired for example overnight.

Usually, the laundry article is immersed in the dry cleaning composition. The amount of dry cleaning composition used and the amount of time the composition contacts the article can vary based on equipment and the number of articles being cleaned. Normally, the dry cleaning process will comprise at least one cycle of contacting the article with a dry cleaning composition and at least one cycle of rinsing the article with a fresh load of dry cleaning solvent.

The mixing of the disposable treatment composition with a cleaning solvent to form a dry cleaning composition may be carried out by any means known in the art. Mixing may be carried out in a separate chamber or in a drum as described below. Preferably, the disposable treatment composition is mixed with a dry cleaning solvent such that the surfactant and/or cleaning agent is effectively dispersed and/or dissolved to obtain the desired cleaning. Suitable mixing devices include pump assemblies or in-line static mixers, a centrifugal pump or other type of pump, a colloid mill or other type of mill, a rotary mixer, an ultrasonic mixer and other means of dispersing one liquid in another, non-miscible liquid can be used to provide effective agitation to cause emulsification.

Static mixers are devices through which the emulsion is passed at high speed and in which said emulsion experiences sudden changes in direction and/or in the diameter of the channels which make up the interior of the mixers. This results in a pressure loss, which is a factor in obtaining a correct emulsion in terms of droplet size and stability.

Preferably, the dry cleaning process is carried out in an automated dry cleaning machine that comprises a closable vessel. The automated dry cleaning machine is preferably closed or sealed in such a way that the dry cleaning solvent can be contained within the machine if needed. The closable vessel usually comprises a drum which can rotate inside said vessel. The laundry articles in need of treatment are placed inside the drum wherein said articles are contacted with the dry cleaning composition. This may be done in any way known in the art such as spraying or even using a mist.

While the laundry articles are in contact with the dry cleaning solvent, it is preferred to add mechanical energy for example by agitating the laundry articles such that cleaning is achieved. Agitation may be carried out by rotating the drum, using ultrasonic energy or other means known in the art.

The volume of the drum is preferably defined as the volume in the drum to which the laundry articles are confined during the agitation. The volume of the drum is meant to describe the volume effectively available for the laundry articles to tumble in and, accordingly, does not include the space taken in by any structures inside the drum. The person skilled in the art can easily determine the volume. For example, for a cylindrical drum the volume can be calculated using the standard formula for cylindrical spaces minus the volume taken in by other structures—if present—such as a baffle, a spray system, a sensor or part of the closed access door. As may be usual with horizontal axis machines, the access door—when closed—may partly extend into the space of the drum. Preferably, the drum is a perforated cylinder which is usually placed inside a larger vessel to which it is rotatably connected. The larger vessel holds the excess cleaning composition not absorbed by the laundry articles.

We have found that some aspects of the present invention may be especially suitable for cleaning a laundry article stained with domestic stain material selected from the group including kitchen grease, particulate soil and mixtures thereof. Therefore, according to one embodiment the dry cleaning process preferably comprises the cycle of contacting a laundry article with a dry cleaning composition whereby the laundry article is stained with domestic stain material selected from kitchen grease, particulate soil and mixtures thereof. Typical particulate soil stains comprise any particulate matter which is capable of staining garments, such as dirt, mud, sand, charcoal, make up, deodorant, toothpaste but also corroded iron particles and mixtures thereof.

We have also found that the ratio of the amount of the dry cleaning composition and the amount of laundry articles said composition is contacted with ( step ii) may be of importance. This ratio is expressed as the liquid to cloth ratio or LCR. Preferably in step ii) the LCR is at most 20, more preferably at most 10, even more preferably at most 7 and preferably greater than 0.5, more preferably greater than 0.7, even more preferably greater than 1 and most preferably greater than 2.5. For the rinse cycle, the same LCR may be used wherein the “liquid” refers to the rinse composition. When the dry cleaning process comprises different cycles, the LCR of each cycle may be different or the same. Preferably, the LCR of each cycle is as described above.

Usually after agitation of the laundry article in the dry cleaning composition, the dry cleaning solvent including any cleaning agents and/or loosened soil will be separated from the laundry articles. This is preferably done by spinning the laundry articles and collecting the dry cleaning composition, although other separation methods known in the art may also be employed such as evaporation. The dry cleaning solvent is then preferably recycled by separating the soil and/or cleaning agents from the solvent.

In other instances it may be advantageous to recirculate at least part of the dry cleaning composition during one cycle. For example by separating a portion of the dry cleaning composition from the laundry articles, optionally filtering soil from the separated portion of dry cleaning composition and contacting the laundry articles with the filtered portion of the dry cleaning composition.

The dry cleaning is usually performed at atmospheric pressure and room temperature, between 10 and 30 □C in most countries. In some instances the process temperature may be elevated to just under the flash point of the most volatile dry cleaning solvent used. Sometimes the process may be performed under reduced or elevated pressure, typically achieved via a vacuum pump or by supplying a gas, such as nitrogen, to the apparatus thereby increasing the pressure the closable vessel. The dry cleaning process may be carried out in any suitable apparatus. Preferably, the apparatus will comprise means to recycle the dry cleaning solvents used to minimise solvent losses into the environment. The dry cleaning composition may be in the form of a micro-emulsion but usually will be in the form of a macro-emulsion, which is generally accepted to be thermodynamically unstable. A suitable process and appliance for dry cleaning is described in U.S. Pat. No. 6,045,588. The solvent will preferably be filtered and recycled in the same appliance. Generally, the laundry articles will be agitated in the dry cleaning process by tumbling, rotating, ultrasonics or any suitable type of mechanical energy (see U.S. Pat. No. 6,045,588).

The surfactants, dry cleaning solvents, cosolvents and cleaning agents which may be used in present invention are described below and may be the same or different for each cycle of the inventive process.

Disposable Treatment Composition

The dry cleaning compositions as described herein are preferably formed by mixing a disposable treatment composition with a dry cleaning solvent.

The disposable treatment composition have any suitable format known in the art. According to one preferred embodiment, the disposable treatment composition is a unit dose composition such as a tablet or, more preferably, a sachet. The sachet may comprise of an outer layer which is soluble in the dry cleaning solvent.

The disposable treatment composition may be dosed from an automatic dosing device from at least one container placed inside the dry cleaning machine or attached to the dry cleaning machine. The amount and—when the disposable treatment composition is dosed from more than one container, the exact composition of the disposable treatment composition—may be varied from wash to wash. This may depend on the user's input for example, by indicating the degree of soiling or type of clothing. In addition or in the alternative, it may depend on an automated system using information located on a label attached to a laundry article that is read by an appropriate sensor. A suitable example includes a radio frequency identification system.

The disposable treatment composition comprises at least one surfactant and/or cleaning agent, optionally a cosolvent, and, optionally, water as described below. The amounts present in the disposable treatment composition are preferably such that after appropriate dosing per kg laundry, the preferred amounts in the dry cleaning composition are obtained as described herein.

The disposable treatment composition may comprise at least one surfactant in amounts of at least 0.1 wt. %, preferably at least 1 wt. %, more preferably at least 25 wt. %, most preferably at least 50 wt. % and at most 99 wt. %, preferably at most 95 wt. % and more preferably at most 90 wt. % and most preferably at most 80 wt. % by weight of the disposable treatment composition. Preferably, the amount of surfactant in the disposable treatment composition is such that in step ii) the surfactant to cloth ratio is at most 0.25, more preferably at most 0.12, more preferably at most 0.08, more preferably at most 0.04, but preferably at least 0.0001, more preferably at least 0.0003, more preferably at least 0.001 and most preferably at least 0.002.

The disposable treatment composition preferably has a flash point of greater than 37.8° C. (100° F.), more preferably greater than 60° C. (140° F.)

When used in the disposable treatment composition, the cleaning agent may be present in amounts of at least 0.001 wt. %, preferably at least 0.1 wt. %, more preferably at least 1 wt. % and at most 90 wt. %, preferably at most 75 wt. % and more preferably at most 40 wt. % by weight of the disposable treatment composition.

With regard to the cosolvent, when used in the disposable treatment composition, the cosolvent may be present in amounts of at least 0.001 wt. %, preferably at least 0.1 wt. %, more preferably at least 1 wt. % and at most 90 wt. %, preferably at most 75 wt. % and more preferably at most 40 wt. % by weight of the disposable treatment composition.

As water is present in the disposable treatment composition, the dry cleaning composition will evidently also contain water. Surprisingly, we have found that the ratio between the amount of water in the dry cleaning composition and the amount of cloth it is contacted with, is important to obtain effective cleaning and garment care. This ratio is expressed as water to cloth ratio (w/w) (WCR) and is described below. Preferably, the water is present in the form of a emulsion or micro-emulsion in the disposable treatment composition.

Dry Cleaning Solvent

The dry cleaning solvent is usually a non-flammable, non-chlorine containing organic dry cleaning solvent. Although the term dry cleaning solvent is used in the singular, it should be noted that a mixture of solvents may also be used. Thus, the singular should be taken to encompass the plural, and vice versa. Because of the typical environmental problems associated with chlorine containing solvents, the solvent preferably does not contain Cl atoms. In addition, the solvent should not be flammable such as most petroleum or mineral spirits having typical flash points as low as 20□C or even lower. The term non-flammable is intended to describe dry cleaning solvents with a flash point of at least 37.8□C, more preferably at least 45□C, most preferably at least 50□C. The limit of a flashpoint of at least 37.8□C for non-flammable liquids is defined in NFPA 30, the Flammable and Combustible Liquids Code as issued by National Fire Protection Association, 1996 edition, Massachusetts USA. Preferred test methods for determining the flash point of solvents are the standard tests as described in NFPA30. One preferable class of solvents is a fluorinated organic dry cleaning solvent including hydrofluorocarbon (HFC) and hydrofluoroether (HFE). However, even more preferred are non flammable non-halogenated solvents. Another class of suitable highly preferred solvents are siloxanes (see below). It should be noted that mixtures of different dry cleaning solvents may also be used.

The most desirable solvents are non-ozone depleting and a useful common definition for the ozone depleting potential is defined by the Environmental Protection Agency in the USA: the ozone depleting potential is the ratio of the impact on ozone of a chemical compared to the impact of a similar mass of CFC-11. Thus, the ODP of CFC-11 is defined to be 1.0.

Hydrofluorocarbons

One preferred hydrofluorocarbon solvent is represented by the formula CₓH_{y}F_{(2x+2-y)}, wherein x is from 3 to 8, y is from 1 to 6, the mole ratio of F/H in the hydrofluorocarbon solvent is greater than 1.6. Preferably, x is from 4 to 6 and most preferred x is 5 and y is 2.

Especially suitable are hydrofluorocarbon solvents selected from isomers of decafluoropentane and mixtures thereof. In particular useful is 1,1,1,2,2,3,4,5,5,5-decafluoropentane. The E.I. Du Pont De Nemours and Company markets this compound under the name Vertrel XF™.

Hydrofluoroethers

Hydrofluoroethers (HFEs) suitable for use in the present invention are generally low polarity chemical compounds minimally containing carbon, fluorine, hydrogen, and catenary (that is, in-chain) oxygen atoms. HFEs can optionally contain additional catenary heteroatoms, such as nitrogen and sulphur. HFEs have molecular structures which can be linear, branched, or cyclic, or a combination thereof (such as alkylcycloaliphatic), and are preferably free of ethylenic unsaturation, having a total of about 4 to about 20 carbon atoms. Such HFEs are known and are readily available, either as essentially pure compounds or as mixtures.

Preferred hydrofluoroethers can have a boiling point in the range from about 40 □C to about 275 □C, preferably from about 50 □C to about 200 □C, even more preferably from about 50 □C to about 121 □C. It is very desirable that the hydrofluoroether has no flashpoint. In general, when a HFE has a flash point, decreasing the F/H ratio or decreasing the number of carbon-carbon bonds each decreases the flash point of the HFE (see WO/00 26206).

Useful hydrofluoroethers include two varieties: segregated hydrofluoroethers and omega-hydrofluoroalkylethers. Structurally, the segregated hydrofluoroethers comprise at least one mono-, di-, or trialkoxy-substituted perfluoroalkane, perfluorocycloalkane, perfluorocycloalkyl-containing perfluoroalkane, or perfluorocycloalkylene-containing perfluoroalkane compound.

HFEs suitable for use in the processes of the invention include the following compounds:
C₄F₉OC₂F₄H, HC₃F₆OC₃F₆H, HC₃F₆OCH₃, C₅F₁₁OC₂F₄H, C₆F₁₃OCF₂H, C₆F₁₃OC₂F₄OC₂F₄H, c-C₆F₁₁CF₂OCF₂H, C₃F₇OCH₂F,
HCF₂O(C₂F₄O)ₙ(CF₂O)ₘCF₂H, wherein m=0 to 2 and n=0 to 3
C₃F₇O[C(CF₃)₂CF₂O]ₚCFHCF₃, wherein p=0 to 5
C₄F₉OCF₂C(CF₃)₂CF₂H, HCF₂CF₂OCF₂C(CF₃)₂CF₂OC₂F₄H, C₇F₁₅OCFHCF₃, C₈F₁₇OCF₂O(CF₂)₅H, C₈F₁₇OC₂F₄OC₂F₄OC₂F₄OCF₂H, C₄F₉OC₂H₅, C₄FₚOCH₃, C₈F₁₇OCH₃.

Preferred HFEs are according to the formula
CₙX₂ₙ₊₁—O—CₘY₂ₘ₊₁

Wherein X and Y are each independently F or H provided that at least one F is present. Preferably, X=F and Y=H;
n=2-15 and m=1-10, but preferably, n=3-8 and m=1-4, or more preferably n=4-6 and m=1-3.

Especially preferred is a HFE wherein n=4 and m=1 or 2 which is marketed under the name of HFE 7100™ and 7200™ respectively by the 3M corporation.

Mixtures of different organic dry cleaning solvents may also be used. For example, a suitable dry cleaning composition may comprise a mixture of HFEs together with a mixture of siloxanes.

When solvent compounds are mentioned, isomers thereof are also included. Thus, suitable HFEs include nonafluoromethoxybutane(C₄F₉OCH₃) isomers such as 1,1,1,2,2,3,3,4,4-nonafluoro-4-methoxy-butane(CH₃OCF₂CF₂CF₂CF₃), 1,1,1,2,3,3-hexafluoro-2-(trifluoromethyl)-3-methoxy-propane(CH₃OCF₂CF(CF₃)₂), 1,1,1,3,3,3-hexafluoro-2-methoxy-2-(trifluoromethyl)-propane(CH₃OC(CF₃)₃), and 1,1,1,2,3,3,4,4,4-nonafluoro-2-methoxy-butane(CH₃OCF(CF₃)CF₂CF₃), approximate isomer boiling point=60□C;

Also isomers of nonafluoroethoxybutane(C₄F₉OC₂H₅) such as 1,1,1,2,2,3,3,4,4-nonafluoro-4-ethoxybutane(CH₃CH₂OCF₂CF₂CF₂CF₃), 1,1,1,2,3, 3-hexafluoro-2-(trifluoromethyl)-3-ethoxypropane(CH₃CH₂OCF₂CF(CF3)₂), 1,1,1,3,3,3-hexafluoro-2-ethoxy-2-(trifluoromethyl)-propane(CH₃CH₂OC(CF₃)₃), and 1,1,1,2,3,3,4,4,4-nonafluoro-2-ethoxybutane(CH₃CH₂OCF(CF₃)CF₂CF₃) with approximate isomer boiling points of 73□C.

Siloxane Dry Cleaning Solvent

Some siloxane solvents may also be used advantageously in the present invention. The siloxane may be linear, branched, cyclic, or a combination thereof. One preferred branched siloxane is tris (trimethylsiloxyl) silane. Also preferred are linear and cyclic oligo dimethylsiloxanes. One preferred class of siloxane solvents is an alkylsiloxane represented by the formula
R₃—Si(—O—SiR₂)w-R

Where each R is independently chosen from an alkyl group having form 1 to 10 carbon atoms and w is an integer from 1 to 30. Preferably, R is methyl and w is 1-4 or even more preferably w is 3 or 4.

Of the cyclic siloxane decamethyl cyclopentasiloxane is particularly effective.

Very useful siloxanes are selected from the group consisting of decamethyl tetrasiloxane, dodecamethyl pentasiloxane and mixtures thereof.

Preferably, the organic solvent is not a terpene. Especially suitable organic dry cleaning solvents include those selected from the group consisting of the isomers of nonafluoromethoxybutane, nonafluoroethoxybutane and decafluoropentane, octamethyl cyclotetrasiloxane, decamethyl cyclopentasiloxane, decamethyl tetrasiloxane, dodecamethyl pentasiloxane and mixtures thereof. Even more preferred are organic dry cleaning solvents include those selected from the group consisting of decamethyl cyclopentasiloxane, decamethyl tetrasiloxane, dodecamethyl pentasiloxane and mixtures thereof.

The dry cleaning compositions of the invention generally contain greater than about 50 percent by weight of organic dry cleaning solvent, preferably greater than about 75 weight percent, more preferably greater than about 80 weight percent, more preferably greater than about 85 weight percent, even more preferably greater than about 95 weight percent, but preferably less than 100 weight percent of organic dry cleaning solvent by weight of the total dry cleaning composition. Such amounts aid in improved drying times and maintain a high flashpoint or no flashpoint at all. For the conditioning cycle the dry cleaning compositions may even comprise of at least 99 weight percent of organic dry cleaning solvent by weight of the total dry cleaning composition and sometimes even 99.999 weight percent of organic dry cleaning solvent.

Water

The dry cleaning process according to one embodiment of the invention may comprise at least one low-aqueous and/or non-aqueous cycle.

For instance, according to yet another aspect of the invention the dry cleaning process comprises:
a) a non-aqueous dry cleaning cycle, wherein
said articles are contacted with a non-aqueous dry cleaning composition comprising
0.001 to 10 wt. % of a surfactant;
0 to 0.01 wt. % of water;
0 to 50 wt. % of a cosolvent and
a non-flammable, non-chlorine containing organic dry cleaning solvent;
b) at least one low-aqueous dry cleaning cycle, wherein said articles are contacted with a low aqueous dry cleaning composition comprising
0.001 to 10 wt. % of a surfactant;
0.01 to 50 wt. % of water;
0 to 50 wt. % of a cosolvent; and
a non-flammable, non-chlorine containing organic dry cleaning solvent;
and, optionally, at least one rinsing cycle, wherein the articles are contacted with
a non-flammable, non-chlorine containing organic dry cleaning solvent.

Depending on the desired cleaning, the low aqueous and non-aqueous compositions may be used in any order. However, in some cases it will be preferred to contact the articles with a non-aqueous composition prior to a low aqueous dry cleaning composition. In fact, the low aqueous dry cleaning cycle may be followed or preceded with various other cycles such as a regeneration, garment care treatment and/or rinsing cycle, and, in fact, any other cycle known to the person skilled in the art.

When water is used in the dry cleaning process, the amount of water present in any cycle of the dry cleaning process is at such a level that laundry articles can be safely cleaned. This includes laundry articles that can only be dry cleaned. The amount of water present in the low aqueous dry cleaning composition is preferably from 0.01 to 10 wt. %, more preferably from 0.01 to 0.9 wt. % water by weight of the dry cleaning composition or more preferably, 0.05 to 0.8 wt. % or most preferable 0.1 to 0.7 wt. %. The amount of water present in the non-aqueous dry cleaning composition is preferably from 0 to 0.1 wt. % water by weight of the dry cleaning composition or more preferably, 0 to 0.01 wt. % or even more preferable 0 to 0.001 wt. % and most preferable 0 wt. %. When the dry cleaning composition comprises water, preferably the water to cloth ratio (w/w) (WCR) is less than 0.45, more preferably less than 0.35, more preferably less than 0.25, more preferably less than 0.2, most preferably less than 0.15, but usually more than 0.0001, preferably more than 0.001, more preferably more than 0.01.

Water may be added separate from the disposable treatment composition. However, when water is added as part of the disposable treatment composition then it is preferred that the amount of water in the disposable treatment composition is such that in step ii) the water to cloth ratio (w/w) is less than 0.15 , preferably less than 0.10 but usually more than 0.0001, preferably more than 0.001, more preferably more than 0.01.

When the dry cleaning process comprises more than one cycle, this WCR preferably applies to all cycles in the dry cleaning process, especially when the dry cleaning composition comprises water and solvent. However, the WCR may or may not differ for each cycle. It is also preferred that this WCR applies to each cycles in the dry cleaning process wherein the LCR is more than 1.

When water is present in the dry cleaning composition or disposable treatment composition, the water is preferably suspended by the surfactant and/or cleaning agent.

Surprisingly, it was found that with specific water to surfactant ratios effective cleaning of for example particulate soil could be obtained while maintaining good garment care. Preferably the water to surfactant ratio (w/w) is at most 25, more preferably at most 10, more preferably at most 5 but preferably at least 0.001, more preferably at least 0.01 and most preferably at least 0.1.

Cosolvents

The dry cleaning compositions and disposable treatment compositions of the invention may contain one or more cosolvents. The purpose of a cosolvent in the dry cleaning compositions of the invention is often to increase the solvency of the dry cleaning composition for a variety of soils. However, if a cosolvent is used the dry cleaning composition is preferably a non-azeotrope as azeotropes may be less robust.

Useful cosolvents of the invention are soluble in the dry cleaning solvent or water, are compatible with typical cleaning agents, and can enhance the solubilisation of hydrophilic composite stains and oils typically found in stains on clothing, such as vegetable, mineral, or animal oils. Any cosolvent or mixtures of cosolvents meeting the above criteria may be used.

Useful cosolvents include alcohols, ethers, glycol ethers, alkanes, alkenes, linear and cyclic amides, perfluorinated tertiary amines, perfluoroethers, cycloalkanes, esters, ketones, aromatics, the fully or partly halogenated derivatives thereof and mixtures thereof. Preferably, the cosolvent is selected from the group consisting of alcohols, alkanes, alkenes, cycloalkanes, ethers, esters, cyclic amides, aromatics, ketones, the fully or partly halogenated derivatives thereof and mixtures thereof.

Representative examples of cosolvents which can be used in the dry cleaning compositions of the invention include methanol, ethanol, isopropanol, t-butyl alcohol, trifluoroethanol, pentafluoropropanol, hexafluoro-2-propanol, methyl t-butyl ether, methyl t-amyl ether, propylene glycol n-propyl ether, propylene glycol n-butyl ether, dipropylene glycol n-butyl ether, propylene glycol methyl ether, ethylene glycol monobutyl ether, trans-1,2-dichloroethylene, decalin, methyl decanoate, t-butyl acetate, ethyl acetate, glycol methyl ether acetate, ethyl lactate, diethyl phthalate, 2-butanone, N-alkyl pyrrolidone (such as N-methyl pyrrolidone, N-ethyl pyrrolidone), methyl isobutyl ketone, naphthalene, toluene, trifluorotoluene, perfluorohexane, perfluoroheptane, perfluorooctane, perfluorotributylamine, perfluoro-2-butyl oxacyclopentane.

Preferably, the cosolvent is present in the compositions of the invention in an effective amount by weight to form a homogeneous composition with the other dry cleaning solvent(s) such as HFE. The effective amount of cosolvent will vary depending upon which cosolvent or cosolvent blends are used and the other dry cleaning solvent(s) used in the composition. However, the preferred maximum amount of any particular cosolvent present in a dry cleaning composition should be low enough to keep the dry cleaning composition non-flammable as defined above.

In general, cosolvent may be present in the dry cleaning compositions of the invention in an amount of from about 1 to 50 percent by weight, preferably from about 5 to about 40 percent by weight, and more preferably from about 10 to about 25 percent by weight. In some exceptional cases the cosolvent may be present in amounts of from about 0.01 percent by weight of the total dry cleaning composition.

Surfactants

The dry cleaning compositions of the invention can utilise many types of cyclic, linear or branched surfactants known in the art, both fluorinated and non-fluorinated. Preferred solvent compatible surfactants include nonionic, anionic, cationic and zwitterionic surfactants having at least 4 carbon atoms, but preferably less than 200 carbon atoms or more preferably less than 90 carbon atoms as described below. Solvent compatible surfactants usually have a solvent-philic part that increases the solubility of the surfactant in the dry cleaning solvent. Effective surfactants may comprise of one or more polar hydrophilic groups and one or more dry cleaning solvent-philic parts having at least 4 carbon atoms so that the surfactant is soluble in said dry cleaning solvent. It is preferred that the surfactant is soluble in the dry cleaning solvent, i.e., to at least the amount of surfactant used in the dry cleaning composition at 20 □C. The composition may comprise one or a mixture of surfactants depending on the desired cleaning and garment care. One preferred surfactant is an anionic surfactant. Another preferred surfactant is a cationic surfactant.

The polar hydrophilic group, Z, can be nonionic, ionic (that is, anionic, cationic, or zwitterionic), or a combination thereof. Typical nonionic moieties include polyoxyethylene and polyoxypropylene moieties. Typical anionic moieties include carboxylate, sulfonate, sulfate, or phosphate moieties. Typical cationic moieties include quaternary ammonium, protonated ammonium, imidazolines, amines, diamines, sulfonium, and phosphbnium moieties. Typical zwitterionic moieties include betaine, sulfobetaine, aminocarboxyl, amine oxide, and various other combinations of anionic and cationic moieties. Especially suitable surfactants comprise at least one polar hydrophilic group Z which is an anionic moiety whereby the counterion may be as described below.

The polar hydrophilic group Z is preferably selected from the group comprising —SO₄M, —SO₃M, —PO₄M₂, —PO₃M₂, —CO₂M and mixtures thereof wherein each M can be independently selected from the group including H, NR₄, Na, K and Li, wherein each R is independently selected from H and C1-4 alkyl radical but preferably H. Preferably M is H but in some cases salts may also be used.

Fluorinated Surfactants

In one preferred embodiment, the surfactant is fluorinated or more preferably a fluorinated acid.

Suitable fluorosurfactants are in most cases those according to the molecular formula (I):
(Xf)ₙ(Y)ₘ(Z)ₚ (I)
and contain one, two or more fluorinated radicals (Xf) and one or more polar hydrophilic groups (Z), which radicals and polar hydrophilic groups are usually (but not necessarily) connected together by one or more suitable linking groups (Y).

Preferably, n and p are integers independently selected from 1 to 4 and m is selected from 0 to 4. When the surfactant comprises more than one Xf, Y or Z group, then each of Xf, Y and Z may be the same or different. Preferably, the polar hydrophilic group is connected by a covalent bond to Y, or in absence of Y, to Xf.

The fluorinated radical, Xf, can generally be a linear or cyclic, saturated or unsaturated, aromatic or non-aromatic, radical preferably having at least 3 carbon atoms. The carbon chain may be linear or branched and may include hetero atoms such as oxygen or sulphur, but preferably not nitrogen. Preferably, Xf is an aliphatic and saturated. A fully fluorinated Xf radical is preferred, but hydrogen or chlorine may be present as substituents provided that not more than one atom of either is present for every two carbon atoms, and, preferably, the radical contains at least a terminal perfluoromethyl group. Radicals containing no more than about carbon atoms are preferred because larger radicals usually represent a less efficient utilisation of fluorine.

Especially suitable Xf groups can be based on perfluorinated carbon: CₙF₂ₙ₊₁—wherein n is from 1-40, preferably 2 to 26, most preferably 2 to 18 or can be based on oligomers of hexafluoropropyleneoxide: [CF(CF₃)—CF₂-O]ₙ wherein n is from 1 to 30. Suitable examples of the latter are marketed by E.I DuPont de Nemours and Co. under the name Krytox™ 157, especially, Krytox™ 157 FSL. Fluoroaliphatic radicals containing about 2 to 14 carbon atoms are more preferred.

The linking group, Y, is selected from groups such as alkyl, alkylene, alkylene oxide, arylene, carbonyl, ester, amide, ether oxygen, secondary or tertiary amine, sulfonamidoalkylene, carboxamidoalkylene, alkylenesulfonamidoalkylene, alkyleneoxyalkylene, or alkylenethioalkylene or mixtures thereof. In one preferred embodiment Y is (CH₂)ₜ or (CH₂)ₜO wherein t is 1 to 10, preferably 1 to 6, most preferably 2 to 4. Alternatively, Y may be absent, in which case Xf and Z are directly connected by a covalent bond.

A particularly useful class of fluoroaliphatic surfactants useful in this invention are those wherein Xf, Y, and Z are as defined, and n is 1 or 2, m is 0 to 2, and p is 1 or 2.

Examples of very useful surfactants are those comprising at least wherein n is 1 to 4, m is 0 to 4, and p is 1 to 4, Z is as defined and
Xf=R¹
Y═(R²)ᵥ
wherein R¹ is a perfluoroalkyl group having 1 to 40 carbon atoms; R² is an alkyl or an alkylene oxide group having 2 to 6 carbon atoms; and v is 0-10

Preferably, the surfactant is according to the formula
[R¹—R²ᵥ]_{w}PO(OH)_{3-w}
wherein R¹ is a perfluoroalkyl group having 1 to 26 carbon atoms; R² is an alkyl or an alkylene oxide group having 2 to 6 carbon atoms; v is 0-10 and w is 1-2.

More preferably, R¹ is a perfluoroalkyl group having 2 to 16 carbon atoms; R² is an alkyl or an alkylene oxide group having 2 to 6 carbon atoms; v is 1 and w is 1 or 2.

Most preferably, R¹ is a perfluoroalkyl group having 2 to 14 carbon atoms; R² is ethylene oxide; v=1 and w=1-2.

Non-Fluorinated Surfactant

One other suitable class of surfactants are non-fluorinated surfactants according the molecular formula (II):
(Xh)ₙ(Y)ₘ(Z)ₚ (II)
wherein Xh is a non-fluorinated radical and (Y), (Z), n, m and p are as described above for molecular formula (I).

Xh may be a linear, branched or cyclic, saturated or unsaturated, aromatic or non-aromatic, radical preferably having at least 4 carbon atoms. Xh preferably includes hydrocarbon and polydimethyl siloxane radicals. When Xh is a hydrocarbon, the carbon chain may be linear, branched or cyclic and may include hetero atoms such as oxygen, nitrogen or sulphur, although in some cases nitrogen is not preferred. Preferably, Xh is aliphatic and saturated. Radicals containing no more than about 24 carbon atoms are preferred. One preferred surfactant is an acid surfactant. Preferred surfactants include anionic surfactants. Anionic surfactants are generally known in the art and include, for example, alkyl aryl sulfonates (such as, for example, alkylbenzenesulfonates), alkyl aryl sulfonic acids (such as, for example, sodium and ammonium salts of toluene-, xylene- and isopropylbenzenesulfonic acids), sulfonated amines and sulfonated amides (such as, for example, amidosulfonates), carboxylated alcohols and carboxylated alkylphenol ethoxylates, diphenyl sulfonates, fatty esters, isothionates, lignin-based surfactants, olefin sulfonates (such as, for example, RCH═CHSO₃Na, where R is C₁₀-C₁₆), phosphorous-based surfactants, protein based surfactants, sarcosine-based surfactants (such as, for example, N-acylsarcosinates such as sodium N-lauroylsarcosinate), sulfates and sulfonates of oils and/or fatty acids, sulfates and sulfonates of ethoxylated alkylphenols, sulfates of alcohols, sulfates of ethoxylated alcohols, sulfates of fatty esters, sulfates of aromatic or fluoro containing compounds, sulfosuccinnamates, sulfosuccinates (such as, for example, diamyl-, dioctyl- and diisobutylsulfosuccinates), taurates, and sulfonic acids.

In one embodiment, preferable surfactants include nitrogen containing materials selected from the group consisting of primary, secondary and tertiary amines, diamines, triamines, ethoxylated amines, amine oxides, amides and betaines, a nonlimiting example of a betaines is Schercotaine materials commercially available from Scher Chemicals and mixtures thereof.

Examples of suitable non-fluorinated anionic surfactants include Crodafos™ 810A (ex Croda) and of suitable succinate surfactants include Aerosol OT (ex Cytec).

Other classes of suitable surfactants include, but are not limited to nonionic and cationic surfactants. Compounds suitable for use as the nonionic surfactant of the present invention are those that carry no discrete charges when dissolved in aqueous media. Nonionic surfactants are generally known in the art and include, for example, alkanol amides (such as, for example, coco, lauric, oleic and stearic monoethanolamides, diethanolamides and monoisopropanolamides), amine oxides (such as, for example, polyoxyethylene ethanolamides and polyoxyethylene propanolamides), polyalkylene oxide block copolymers (such as, for example, poly(oxyethylene-co-oxypropylene)), ethoxylated alcohols, (such as, for example, isostearyl polyoxyethylene alcohol, lauryl, cetyl, stearyl, oleyl, tridecyl, trimethylnonyl, isodecyl, tridecyl), ethoxylated alkylphenols (such as, for example, nonylphenol), ethoxylated amines and ethoxylated amides, ethoxylated fatty acids, ethoxylated fatty esters and ethoxylated fatty oils (such as, for example, mono- and diesters of acids such as lauric, isostearic, pelargonic, oleic, coco, stearic, and ricinoleic, and oils such as castor oil and tall oil), fatty esters, glycerol esters (such as, for example, glycerol monostearate, glycerol monolaurate, glycerol dilaurate, glycerol monoricinoleate, and glycerol oleate), glycol esters (such as, for example, propylene glycol monostearate, ethylene glycol monostearate, ethylene glycol distearate, diethylene glycol monolaurate, diethylene glycol monolaurate, diethylene glycol monooleate, and diethylene glycol stearate), lanolin-based surfactants, monoglycerides, phosphate esters, polysaccharide ethers, propoxylated fatty acids, propoxylated alcohols, and propoxylated alkylphenols, butoxylated fatty acids, butoxylated alcohols, and butoxylated alkylphenols, protein-based organic surfactants, sorbitan-based surfactants (such as, for example, sorbitan oleate, sorbitan monolaurate, and sorbitan palmitate), sucrose esters and glucose esters, and thio- and mercapto-based surfactants.

Other examples of suitable non-fluorinated surfactants include polymers such as—but not limited to—a silicone-containing polymer functionalized with hydrogen bonding substituents selected from the group consisting of polyoI substituents, polyamine substituents, alkanolamine substituents, and combinations thereof. Said silicone-containing polymer is preferably further functionalized with at least one optional unit selected from the group consisting of monoamines, monoalcohols, polyaIkylene oxides, amides, and combinations thereof.

Other particularly suitable polymers are polyoxyalkylene polymers comprising C4 or higher molecular weight alkylene oxide monomeric units, C1 or higher molecular weight glycidyl ether monomeric units, or combinations thereof and having a solubility at room temperature of at least 0.5 % by weight in the lipophilic fluid the polymer is to be used with, preferably polyoxyalkylene polymer comprising butylene oxide monomer units.

Said polyoxyalkylene polymer functionalized with polar, hydrophilic or both polar and hydrophilic substituents selected from the group consisting of alcohols, polyoIs, amines, polyamines, alkanolamines, ethoxylated and/or propoxylated amines, amides, ethoxylated and/or propoxylated amides, polyamides, urethanes and polyurethanes, oxyethylene, polyoxyethylenes, oxypropylene, polyoxypropylenes, carboxylic acids and salts, aminocarboxylates, amidocarboxylates, mono and diphosphate esters, phosphonates, amino phosphonates, monosulfates, sulfonates, amine oxides, quaternized amines, phosphine oxides, phenols, polyfunctional chelant groups, and combinations thereof.

In a preferred embodiment, one component of the present invention comprises one or more nonionic surfactants according to one or more of the structural formulas III and IV:
R⁹—O—(CH₂—CH₂—O)ₙ—R¹⁰ (III)
R⁹—O—(CH₂—C(CH₃)H—O)ₙ—R¹⁰ (IV)
wherein:
R⁹ is a monovalent hydrocarbon group of from 1 to 30 carbons that may be linear, cyclic, branched, unsaturated or aromatic;
R¹⁰ is hydrogen or a monovalent hydrocarbon group of 1 to 30 carbons that may be linear, cyclic, branched, unsaturated, aromatic or fluoro containing; and n is from about 1 to about 100, more preferably from about 1 to about 40. In a highly preferred embodiment, R⁹ contains from 2 to about 24 carbons, even more preferably from 8 to 24 carbons, R¹⁰ is H and n is from about 2 to about 20.

Other suitable nonionic surfactants include polyethylene oxide condensates of nonyl phenol and myristyl alcohol, such as in U.S. Pat. No. 4,685,930 Kasprzak; and b) fatty alcohol ethoxylates, R—(OCH₂CH₂)ₐOH wherein a=1 to 100, typically 1 to 30, R=hydrocarbon residue 8 to 20 C atoms, typically linear alkyl. Examples polyoxyethylene lauryl ether, with 4 or 10 oxyethylene groups; polyoxyethylene cetyl ether with 2, 6 or 10 oxyethylene groups; polyoxyethylene stearyl ether, with 2, 5, 15, 20, 25 or 100 oxyethylene groups; polyoxyethylene (2), (10) oleyl ether, with 2 or 10 oxyethylene groups. Commercially available examples include, but are not limited to: BRIJ and NEODOL. See also U.S. Pat. No. 6,013,683 Hill et al. Other suitable nonionic surfactants include Tween™.

Especially preferred nonionic surfactants are characterized by an HLB of from 6 to about 11, preferably from about 6.5 to about 9.5, and more preferably from about 7 to about 9. Nonlimiting examples of commercially available preferred surfactants are Neodol 91-2. 5 (HLB=8.5), Neodol 23-3 (HLB=7. 9) end Neodol 25-3 (HLB=7.5).

Suitable cationic surfactants include, but are not limited to dialkyldimethyl ammonium salts having the formula:
R′R″N³⁰ (CH₃)₂X⁻ wherein R′ and R″ are each independently selected from the group consisting of hydrocarbon containing moiety containing 1-30 C atoms or derived from tallow, coconut oil or soy, X═Cl, I or Br. Examples include: didodecyldimethyl ammonium bromide (DDAB), dihexadecyldimethyl ammonium chloride, dihexadecyldimethyl ammonium bromide, dioctadecyldimethyl ammonium chloride, dieicosyldimethyl ammonium chloride, didocosyldimethyl ammonium chloride, dicoconutdimethyl ammonium chloride, ditallowdimethyl ammonium bromide (DTAB). Commercially available examples include, but are not limited to: ADOGEN, ARQUAD, TOMAH, VARIQUAT. See also U.S. Pat. No. 6,013,683 Hill et al.

Also suitable surfactants are silicone surfactants including, but not limited to the polyalkyleneoxide polydimethylsiloxanes having a polydimethylsiloxane hydrophobic moiety and one or more hydrophilic polyalkyleneoxide side chains and have the general formula:
R¹¹—(CH₃)₂SiO—[(CH₃)₂SiO]ₐ—[(CH₃)(R¹¹)SiO]_{b}—Si(CH₃)₂—R¹¹
wherein a+b are from about 1 to about 50, preferably from about 3 to about 30, more preferably from about 10 to about 25, and each R¹¹ is the same or different and is selected from the group consisting of methyl and a poly(ethyleneoxide/propyleneoxide)copolymer group having the general formula:
—(CH₂)ₙO(C₂H₄O)_{c}(C₃H₆O)dR¹²
with at least one R¹¹ being a poly(ethyleneoxide/propyleneoxide)copolymer group, and wherein n is 3 or 4, preferably 3; total c (for all polyalkyleneoxide side groups) has a value of from 0 to about 100, preferably from about 6 to about 100; total d is from 0 to about 14, preferably from 0 to about 3; and more preferably d is 0; total c+d has a value of from about 5 to about 150, preferably from about 9 to about 100 and each R¹² is the same or different and is selected from the group consisting of hydrogen, an alkyl having 1 to 4 carbon atoms, and an acetyl group, preferably hydrogen and methyl group. Examples of these surfactants may be found in U.S. Pat. No. 5,705,562 and U.S. Pat. No. 5,707,613, both of which are incorporated herein by reference.

Examples of this type of surfactants are the Silwet™ surfactants which are available from CK Witco, OSi Division, Danbury, Connecticut. Representative Silwet™ surfactants are for example L-7608, L-7607, L-77, L-7605, L-7604, L-7600, L-7657, L-7602. The molecular weight of the polyalkyleneoxide group (R¹¹) is less than or equal to about 10,000. Preferably, the molecular weight of the polyalkyleneoxide group is less than or equal to about 8,000, and most preferably ranges from about 300 to about 5,000. Thus, the values of c and d can be those numbers which provide molecular weights within these ranges. However, the number of ethyleneoxide units (—C₂H₄O) in the polyether chain (R¹¹) must be sufficient to render the polyalkyleneoxide polysiloxane water dispersible or water soluble. If propyleneoxide groups are present in the polyalkyleneoxide chain, they can be distributed randomly in the chain or exist as blocks. Especially preferred Silwet™ surfactants are L-7600, L-7602, L-7604, L-7605, L-7657, and mixtures thereof. Besides cleaning and/or emulsifying activity, polyalkyleneoxide polydimethylsiloxane surfactants can also provide other benefits, such as anti-static benefits, and softness to fabrics.

The preparation of polyalkyleneoxide polydimetylsiloxanes is well known in the art. Polyalkyleneoxide polydimethylsiloxanes of the present invention can be prepared according to the procedure set forth in U.S. Pat. No. 3,299,112, incorporated herein by reference.

Another suitable silicone surfactant is SF-1488, which is available from GE silicone fluids. Especially preferred silicone surfactants include Tegopren™ 7008 and 7009 (ex Goldschmidt).

These and other surfactants suitable for use in combination with the organic dry cleaning solvent as adjuncts are well known in the art, being described in more detail in Kirk Othmer's Encyclopaedia of Chemical Technology, 3rd Ed., Vol. 22, pp. 360-379, “Surfactants and Detersive Systems”, incorporated by reference herein. Further suitable nonionic detergent surfactants are generally disclosed in U.S. Pat. No. 3,929,678, Laughlin et al., issued Dec. 30, 1975, at column 13, line 14 through column 16, line 6, incorporated herein by reference. Other suitable detergent surfactants are generally disclosed in WO-A-0246517.

The surfactant or mixture of surfactants is present in a cleaning effective amount. A cleaning effective amount is the amount needed for the desired cleaning. This will, for example, depend on the number of articles, level of soiling and volume of dry cleaning composition used. However, surprisingly effective cleaning was observed when the surfactant was present from at least 0.001 wt. % to 10 wt. % by weight of the dry cleaning composition. More preferably, the surfactant is present from 0.01 to 3 wt. % or even more preferably from 0.05 to 0.9 wt. % by weight of the dry cleaning composition. More preferably, the surfactant is present from 0.1 to 0.8 wt. % or even more preferably from 0.3 to 0.7 wt. % by weight of the dry cleaning composition.

Surprisingly, it was found that the surfactant to cloth ratio (w/w) (SCR) was important in many cases to obtain an effective cleaning while maintaining a good garment care. Preferably, the SCR is at most 0.25, more preferably at most 0.12, more preferably at most 0.08, more preferably at most 0.04, but preferably at least 0.0001, more preferably at least 0.0003, more preferably at least 0.001 and most preferably at least 0.002.

Optional Cleaning Agents

The compositions may contain one or more optional cleaning agents. Cleaning agents include any agent suitable for enhancing the cleaning, appearance, condition and/or garment care. Generally, the cleaning agent may be present in the dry cleaning composition in an effective amount, i.e., an amount needed to obtain the desired effect, or preferably of about 0 to 20 wt. %, preferably 0.001 wt. % to 10 wt. %, more preferably 0.01 wt. % to 2 wt. % by weight of the total dry cleaning composition.

Some suitable cleaning agents include, but are not limited to builders, enzymes, bleach activators, bleach catalysts, bleach boosters, bleaches, alkalinity sources, antibacterial agents, colorants, perfumes, pro-perfumes, finishing aids, lime soap dispersants, composition odor control agents, polymeric dye transfer inhibiting agents, crystal growth inhibitors, photobleaches, heavy metal ion sequestrants, anti-tarnishing agents, anti-microbial agents, anti-oxidants, anti-redeposition agents, soil release polymers, electrolytes, pH modifiers, thickeners, abrasives, divalent or trivalent ions, metal ion salts, enzyme stabilisers, corrosion inhibitors, diamines or polyamines and/or their alkoxylates, suds stabilising polymers, process aids, fabric softening agents, optical brighteners, hydrotropes, suds or foam suppressors, suds or foam boosters, anti-static agents, dye fixatives, dye abrasion inhibitors, wrinkle reduction agents, wrinkle resistance agents, soil repellency agents, sunscreen agents, anti-fade agents, and mixtures thereof.

Suitable odor control agents, which may optionally be used as finishing agents, include cyclodextrins, odor neutralisers, odor blockers and mixtures thereof. Suitable odor neutralisers include aldehydes, flavanoids, metallic salts, water-soluble polymers, zeolites, activated carbon and mixtures thereof.

Perfumes and perfumery ingredients useful in the compositions of the present invention comprise a wide variety of natural and synthetic chemical ingredients: including, but not limited to, aldehydes, ketones, esters, and the like. Also included are various natural extracts and essences which can comprise complex mixtures of ingredients, such as orange oil, lemon oil, rose extract, lavender, musk, patchouli, balsamic essence, sandalwood oil, pine oil, cedar, and the like. Finished perfumes may comprise extremely complex mixtures of such ingredients. Pro-perfumes are also useful in the present invention. Such materials are those precursors or mixtures thereof capable of chemically reacting, e.g., by hydrolysis, to release a perfume, and are described in patents and/or published patent applications to Procter and Gamble, Firmenich, Givaudan, Quest, IFF, Unilever and others.

The invention will now be illustrated by way of the following non-limiting example.

### EXAMPLE

In this example, the fabric care effect of the water content of the dry cleaning composition used in the process of the invention on various types of test cloths treated therewith was determined. More in particular, the effect of said water content on the shrinkage of these test cloths was observed.

Several types of test cloths (cotton, silk, and rayon) were marked with 4 points located at corners of an imaginary square having a rib size of 20 cm.

These test cloths were washed at ambient temperature with a water/oil emulsion consisting of 1% wt water, 0.5% wt silicone surfactant (Tegopren 7008, ex Goldschmidt) and 98.5% wt of decamethyl cyclopentasiloxane (D5) dry cleaning solvent. These wash cycles were carried out using varying Water-to-Cloth Ratios (WCR's; expressed as gram water/gram cloth) of 0.05, 0.1, and 0.15. These WCR's were obtained by applying different amounts of the water/oil emulsion in each wash cycle.

Furthermore, the test cloths were also washed with an emulsion consisting of 99.5% wt of D5 dry cleaning solvent and 0.5% wt of Tegopren 7008, so as to obtain a shrinkage result for a WCR of 0. In this last-mentioned case, a Liquid-to-Cloth Ratio (LCR, expressed as gram emulsion/gram test cloth) of 5 was applied.

After each wash cycle, the test cloths were rinsed with D5 using a LCR of, and then tumble dried.

Data on shrinkage were obtained by measuring the changes in distance between the 4 points on each test cloth resulting from the above-described dry cleaning treatment.

The following shrinkage results (expressed as %-change of the area between the points on the tested cloths) were obtained for the various WCR's applied:

| | Shrinkage results | | |
|---|---|---|---|
| WCR | Cotton | Silk | Rayon |
| 0 | 1 | 1 | 0.5 |
| 0.05 | 2.5 | 1 | 1 |
| 0.1 | 6-8 | 6-7 | 5 |
| 0.15 | 7 | 8 | 7 |

It is noted that fabric shrinkage is generally acceptable if the % area change as a result of the dry-cleaning treatment is less than 5%.

In view of the above shrinkage results, it can therefore be concluded that an amount of water in the water/oil emulsion which corresponds to a WCR of 0.05 is acceptable whereas an amount of water in said emulsion corresponding to a WCR of 0.10 is unacceptable. It follows that the amount of water should not be higher than ca. 60 ml water/kg laundry for ensuring to obtain acceptable shrinkage results.

The various test cloths were also used for determining the effect on another fabric care effect of the above dry cleaning method, i.e. the effect on wrinkling. Also in this case, it was found that an amount of water in the water/oil emulsion corresponding to a WCR of 0.1 is just too high for obtaining acceptable results.

In view of these results on the fabric care effects of water used in dry cleaning, it follows that the amount of water in the disposable treatment composition used in the process of the invention should be less than 100 ml/kg laundry.

1. An in-home dry cleaning process carried out in a dry-cleaning machine containing a non-flammable, non-chlorine containing organic dry cleaning solvent, and comprising the steps of
(i) mixing said solvent with a disposable treatment composition to form a dry cleaning composition; and subsequently
(ii) contacting a laundry article with the dry cleaning composition,
wherein said disposable treatment composition contains
(a) an effective amount of at least one surfactant and/or cleaning agent;
(b) an amount of water (expressed as ml water/kg laundry) of less than 100, preferably less than 50, more preferably less than 3, and more than 0.01, preferably more than 0.05, more preferably more than 0.1, wherein said amount of water is sufficient to give both good cleaning performance and effective fabric care.

2. An in-home dry cleaning process according to claim 1, whereby said disposable treatment composition comprises at least one surfactant in amounts of at least 0.1 wt. %, preferably at least 1 wt. %, more preferably at least 25 wt. %, most preferably at least 50 wt. % and at most 99 wt. %, preferably at most 95 wt. % and more preferably at most 90 wt. % and most preferably at most 80 wt. % by weight of the disposable treatment composition.

3. An in-home dry cleaning process according to claim 1, whereby said. disposable treatment composition comprises at least one cleaning agent in amounts of at least 0.001 wt. %, preferably at least 0.1 wt. %, more preferably at least 1 wt. % and at most 90 wt. %, preferably at most 75 wt. % and more preferably at most 40 wt. % by weight of the disposable treatment composition.

4. An in-home dry cleaning process according to claim 1, whereby said disposable treatment composition comprises a cosolvent in amounts of at least 0.001 wt. %, preferably at least 0.1 wt. %, more preferably at least 1 wt. % and at most 90 wt. %, preferably at most 75 wt. % and more preferably at most 40 wt. % by weight of the disposable treatment composition.

5. An in-home dry cleaning process according to claim 1, whereby said disposable treatment composition comprises a non-flammable, non-chlorine containing organic dry cleaning solvent in amounts of at least 0.001 wt. %, preferably at least 0.1 wt. %, more preferably at least 1 wt. % and at most 90 wt. %, preferably at most 75 wt. % and more preferably at most 40 wt. % by weight of the disposable treatment composition.

6. An in-home dry cleaning process according to claim 1, whereby the amount of surfactant in the disposable treatment composition is such that in step ii) the surfactant to cloth ratio is at most 0.25, more preferably at most 0.12, more preferably at most 0.08, more preferably at most 0.04, but preferably at least 0.0001, more preferably at least 0.0003, more preferably at least 0.001 and most preferably at least 0.002.

7. An in-home dry cleaning process according to claim 1, whereby all the water in the dry cleaning composition is provided by the disposable treatment composition.

8. An in-home dry cleaning process according to claim 1, whereby in step ii) the liquid to cloth ratio is at most 20, more preferably at most 10, even more preferably at most 7 and preferably greater than 0.5, more preferably greater than 0.7, even more preferably greater than 1 and most preferably greater than 2.5.
